(19) United States
(12) Patent Application Publication
(10) Pub. No.: US 2021/0369669 A1
(43) Pub. Date: Dec. 2, 2021

(54) AUTOPHAGY ACTIVATORS AND INHIBITORS OF FERROPTOSIS FOR PREVENTING ACUTE RENAL FAILURE AND NEUROTOXCITY INDUCED BY CERTAIN ANTIBIOTICS

(71) Applicant: SystaMedic Inc., Stonington, CT (US)

(72) Inventors: Anton Franz Joseph FLIRI, Stonington, CT (US); Palaniyandi MANIVASAKAM, West Roxbury, MA (US); Joyce SUTCLIFFE, Westbrook, CT (US)

(73) Assignee: SystaMedic Inc., Stonington, CT (US)

(21) Appl. No.: 17/290,510

(22) PCT Filed: Oct. 18, 2019
(86) PCT No.: PCT/US19/56855

§ 371 Date: Apr. 30, 2021

Related U.S. Application Data
(60) Provisional application No. 62/753,292, filed on Oct. 31, 2018.

(51) Int. Cl. A61K 31/381; A61K 31/7036; A61K 38/12; A61K 31/7048; A61K 31/65; A61K 39/395; A61K 31/675; A61P 13/12

(52) U.S. Cl. CPC A61K 31/381; A61K 31/7036; A61K 38/12; A61P 13/12; A61K 31/65; A61K 39/3955; A61K 31/675; A61K 31/7048

(57) ABSTRACT

The present disclosure relates to pharmaceutical compositions, pharmaceutical combinations and methods of treatment including zileuton, edaravone and atorvastatin compounds combined with nephrotoxicity-inducing antibiotic or anticancer drugs for treating bacterial infections and cancers.

### CROSS-REFERENCE TO RELATED APPLICATIONS

This application claims priority to and the benefit of U.S. Provisional Patent Application Ser. No. 62/753,292 filed Oct. 31, 2018, the disclosure of which is incorporated herein by reference in its entirety.

### FIELD

This disclosure relates to pharmaceutical compositions, pharmaceutical combinations and methods of treatment of bacterial infections and cancer.

### BACKGROUND

Sepsis is a clinical condition occurring in patients following infection or injury and it remains a major challenge in intensive care units. Acute kidney injury (AKI) sometimes called acute kidney failure or acute renal failure is a sudden episode of kidney failure or kidney damage that happens within a few hours or a few days and causes a build-up of waste products in the blood. AKI can also affect other organs such as the brain, heart, and lungs and is common in patients who are in the hospital, in intensive care units, and especially in older adults. Treatments would be initiated at the time of the earliest detection of a build-up of waste products in the blood or when patients are subjected to treatments/conditions known to be causing AKI such as bacterial infections. Acute renal failure occurs in approximately 19 percent of patients with moderate sepsis and in 51 percent of patients with septic shock when blood cultures are positive. In the United States an estimated 700,000 sepsis cases occur every year causing 210,000 fatalities. The combination of acute renal failure and sepsis is associated with 70 percent mortality and exceeds the number of deaths caused by myocardial infarction (See, Schrier R. W. 2004). In addition, several anticancer drugs and antibiotics such as cisplatin, ifosfamide, neomycin, kanamycin, paromomycin, bacitracin, the polymyxins (polymyxin B, polymyxin B Sulfate, colistin, colistin sulfomethate, sodium colistimethate), amphotericin B and tetracyclines are known to induce therapy-limiting renal toxicity and neurotoxicity. The side effects are particularly worrisome in the case of bacterial infections requiring treatment with polymyxin B and colistin which are currently used as “last-line” treatment against multidrug-resistant Gram-negative bacteria infections (See, Dalfino L. et al. 2012). Thus, the nephrotoxicity of colistin and polymyxin B (and to a much lesser degree its neurotoxicity; See, Nation R. L. 2016) is dose-limiting as the plasma polymyxin concentrations associated with renal damage overlap those required for antibacterial effects. Thus, in the case of polymyxin B and colistin treatment, renal toxicity is observed with the IV administration of the recommended adult maximum loading dose of 300-360 mg colistin base activity (CBA) (˜9-10.6 million IU), followed by a dose 150 mg CBA 12-24 hours later and maintaining a dose of 300 mg CBA in divided doses daily (See, Tsuji B. T. et al. 2019, which is hereby incorporated by reference) wherein 1 million IU corresponds to ˜33 mg CBA, and 1 million IU also corresponds to ˜80 mg of the chemical colistin methanesulfonate. The recommended loading dose of polymyxin B is 2.0-2.5 mg/kg (equivalent to 20,000 to 25,000 IU/kg) based on total body weight (TBW) and a maintenance dose of 1.25 to 1.5 mg/kg (equivalent to 12,500 to 15,000 IU/kg TBW) every 12 hours wherein 10,000 International Units of polymyxin equals 1 mg and in case of polymyxin B sulfate, which is the sulfate salt of polymyxins B1 and B2, 1 mg of the anhydrous material has a potency of not less than 6000 polymyxin B IU per mg. Likewise renal impairment is observed with the administration of the recommended daily adult human doses of 300 mg CBA and 3 mg/kg polymyxin B. Despite this adverse event, intravenous and aerosolized polymyxins are being used increasingly for treating patients with infections caused by multidrug-resistant Gram-negative bacteria including Acinetobacter baumannii, Pseudomonas aeruginosa, and Enterobacteriaceae (including carbapenem-resistant strains) (See, Clin Med Res. 2006 June; 4(2): 138-146.) In addition to their bactericidal effect, polymyxins can bind and neutralize lipopolysaccharides (LPS), also known as lipoglycans and endotoxins and may reduce the pathophysiologic effects of endotoxin in the circulation and could serve to attenuate sepsis (See, Giacometti A 2003). While the nephrotoxicity of polymyxin drugs appears to be reversible upon cessation of antibiotic treatment, this mitigating measure becomes life-threatening in the case of drug-resistant bacteria infections. Since induction of renal toxicity by sepsis and polymyxins appear to involve similar molecular mechanisms, patients with sepsis are particularly sensitive to exacerbation of renal injury caused by polymyxin B or colistin treatments. Thus, compositions and methods that allow administration of therapeutic doses of renal injury-causing antibiotics such as neomycin, kanamycin, gentamicin, paromomycin, bacitracin, the polymyxins (polymyxin B, polymyxin B sulfate, colistin, colistin sulfomethate, sodium colistimethate), amphotericin B and tetracyclines are particularly clinically useful.

### SUMMARY

In one embodiment, a method of treating bacterial infections in a mammal is provided. The method includes administering to said mammal in need of such treatment an effective amount of zileuton or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a combination is provided. The combination includes (a) zileuton or a pharmaceutically acceptable salt thereof and (b) one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a pharmaceutical composition for treating bacterial infections in a mammal is provided. The pharmaceutical includes zileuton or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate MRX-8, SPR741, SPR206, CA824, FADDI-002, FADDI-003, FADDI-287, MICuRx-12, NAB739, NAB815, octapeptin C4; and a pharmaceutically acceptable carrier.

In another embodiment a method of treating cancer in a mammal or prolonging the survival of the mammal is provided. The method includes administering to said mammal in need of such treatment an effective amount of zileuton or a pharmaceutically acceptable salt thereof and an effective amount of a nephrotoxicity- or neurotoxicity-inducing anticancer drug selected from the group consisting of ifosfamide, ipilimumab, pembrolizumab and nivolumab.

In another embodiment a combination is provided. The combination includes (a) zileuton or a pharmaceutically acceptable salt thereof and (b) one or more nephrotoxicity- or neurotoxicity-inducing anticancer drugs selected from the group consisting of ifosfamide, ipilimumab, pembrolizumab and nivolumab.

In another embodiment a pharmaceutical composition for treating cancer in a mammal or prolonging the survival of the mammal is provided. The pharmaceutical composition includes zileuton or a pharmaceutically acceptable salt thereof, preferably an effective amount thereof; one or more nephrotoxicity- or neurotoxicity-inducing anticancer drugs, preferably an effective amount thereof, selected from the group consisting of ifosfamide, ipilimumab, pembrolizumab and nivolumab; and a pharmaceutically acceptable carrier.

In another embodiment, a method of treating acute kidney injury in a mammal is provided. The method includes administering to said mammal in need of such treatment of an effective amount of zileuton or a pharmaceutically acceptable salt thereof.

In another embodiment, a method of treating bacterial infections in a mammal is provided. The method includes administering to said mammal in need of such treatment an effective amount of edaravone or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a combination is provided. The combination includes (a) edaravone or a pharmaceutically acceptable salt thereof and (b) one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a pharmaceutical composition for treating bacterial infections in a mammal is provided. The pharmaceutical includes edaravone or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, octapeptin C4; and a pharmaceutically acceptable carrier.

In another embodiment a method of treating cancer in a mammal or prolonging the survival of the mammal is provided. The method includes administering to said mammal in need of such treatment an effective amount of edaravone or a pharmaceutically acceptable salt thereof and an effective amount of a nephrotoxicity- or neurotoxicity-inducing anticancer drug selected from the group consisting of ifosfamide, ipilimumab, pembrolizumab and nivolumab.

In another embodiment a combination is provided. The combination includes (a) edaravone or a pharmaceutically acceptable salt thereof and (b) one or more nephrotoxicity- or neurotoxicity-inducing anticancer drugs selected from the group consisting of ifosfamide, ipilimumab, pembrolizumab and nivolumab.

In another embodiment a pharmaceutical composition for treating cancer in a mammal or prolonging the survival of the mammal is provided. The pharmaceutical composition includes edaravone or a pharmaceutically acceptable salt thereof, preferably an effective amount thereof; one or more nephrotoxicity- or neurotoxicity-inducing anticancer drugs, preferably an effective amount thereof, selected from the group consisting of ifosfamide, ipilimumab, pembrolizumab and nivolumab; and a pharmaceutically acceptable carrier.

In one embodiment, a method of treating bacterial infections in a mammal is provided. The method includes administering to said mammal in need of such treatment an effective amount of atorvastatin or a pharmaceutically acceptable salt thereof and an effective amount of a nephrotoxicity- or neurotoxicity-inducing antibiotic selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a combination is provided. The combination includes (a) atorvastatin or a pharmaceutically acceptable salt thereof and (b) one or more nephrotoxicity- or neurotoxicity-inducing antibiotics selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

In another embodiment a pharmaceutical composition for treating bacterial infections in a mammal is provided. The pharmaceutical includes atorvastatin or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate MRX-8, SPR741, SPR206, CA824, FADDI-002, FADDI-003, FADDI-287, MICuRx-12, NAB739, NAB815 and octapeptin C4; and a pharmaceutically acceptable carrier.

In another embodiment a method of treating cancer in a mammal or prolonging the survival of the mammal is provided. The method includes administering to said mammal in need of such treatment an effective amount of atorvastatin or a pharmaceutically acceptable salt thereof and an effective amount of a nephrotoxicity- or neurotoxicity-inducing anticancer drug selected from the group consisting of ifosfamide, ipilimumab, pembrolizumab and nivolumab.

In another embodiment a combination is provided. The combination includes (a) atorvastatin or a pharmaceutically acceptable salt thereof and (b) one or more nephrotoxicity- or neurotoxicity-inducing anticancer drugs selected from the group consisting of ifosfamide, ipilimumab, pembrolizumab and nivolumab.

In another embodiment a pharmaceutical composition for treating cancer in a mammal or prolonging the survival of the mammal is provided. The pharmaceutical composition includes atorvastatin or a pharmaceutically acceptable salt thereof, preferably an effective amount thereof; one or more nephrotoxicity- or neurotoxicity-inducing anticancer drugs, preferably an effective amount thereof, selected from the group consisting of ifosfamide, ipilimumab, pembrolizumab and nivolumab; and a pharmaceutically acceptable carrier.

### DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustration of the effect of oxidative stress on mitophagy and ferroptosis;

FIG. 2 is a graphic illustration of the urea levels data from the rat experimental; and

FIG. 3 is a graphic illustration of the creatinine levels data from the rat experimental.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The aspects of the present disclosure relate to combinations, pharmaceutical compositions and methods of treatment using them including zileuton, edaravone or atorvastatin and one or more nephrotoxicity- or neurotoxicity-inducing antibiotics, or anticancer, drugs. The aforementioned methods can include a method of treating sepsis and acute renal injury.

It is known that sepsis and polymyxin-induced renal failure is associated with apoptosis of kidney tubular cells. (See, Azad M. A, et al. 2013). Similarly, the induction of apoptosis-related processes plays a key role in the observed neurotoxicity of polymyxin antibiotics (See, Ajiboye T. O. 2018). Elderly patients with diabetes mellitus and chronic kidney disease seem to be at particularly high risk for development of medication-induced AKI, and multiple studies have shown that diabetes alone is an independent risk factor for development of acute kidney injury. Since the concise molecular mechanism determining relationships between these observations are not known we used a proprietary technology (See, CN 108604221 A 2018) for pinpointing key proteins involved. The result of this analysis, shown in Table 1, reveals that certain antibiotics affect proteins involved in modulating molecular processes that become activated by the body's response to oxidative stress (See, Linkermann A., et al. 2014). Functional relationships between proteins listed in Table 1 can be ascertained using the String database (Szklarczyk D, et al. The STRING database in 2017: quality-controlled protein-protein association networks, made broadly accessible. Nucleic Acids Res. 2019 Jan. 8; 47(D1):D607-D613. doi: 10.1093/nar/gky1131.).

TABLE 1

| | | Staph. | Patho- | | | | |
|---|---|---|---|---|---|---|---|
| | response to | aureus | physiology | | | | |
| | oxidative | induced | of acute | Neuro- | | response to | Diabetic |
| Proteins | stress | sepsis | renal injury | toxicity | mitophagy | antibiotics | Nephropathy |
| CASP3 | y | | y | y | y | y | y |
| HMOX1 | y | y | y | y | y | y | y |
| JUN | y | | y | | y | y | y |
| PPIF | y | | y | | | y | |
| FAS | y | | y | | y | | y |
| TNF | y | y | y | | y | | y |
| HGF | | | y | | | | |
| TLR4 | y | y | y | y | y | | y |
| LCN2 | | y | y | | | | y |
| FGF23 | y | | y | | | | y |
| MET | | | y | | y | | y |
| BAD | y | | | | y | y | |
| BCL2 | y | | | | y | y | y |
| BECN1 | y | | | y | y | y | y |
| BNIP3 | y | | | y | y | y | y |
| PIK3CA | y | | | | | y | |
| PRKCD | y | | | | | y | |
| NFE2L2 | y | y | | y | y | y | y |
| MAP1LC3A | y | | | y | y | y | y |
| MT-CYB | y | y | | | | y | |
| BNIP3L | y | | | | | | |
| BAX | | | | | | | y |
| BID | | | | | | | y |
| ITGB3BP | | | | | | | |
| SQSTM1 | | | | | y | | y |
| CDK5 | y | y | y | y | y | | |
| TP53 | y | y | y | y | y | | y |
| DRP1 | y | y | y | y | y | y | y |

Consistent with this assessment, antioxidant approaches (e.g., ascorbic acid) have shown promising results in protecting the kidney of rodents exposed to colistin, yet none of these strategies have yet reached patients (See, Gai Z., et al. 2019). Among the molecular processes protecting cells against oxidative stress is a process termed mitophagy which, for example, is activated as early as 3 hours after induction of sepsis (See, Hsiao H. W. et al. 2012). The relevance of mitophagy for protecting kidney functions is evidenced by the observation that the time-dependent decline in mitophagy is associated with proximal tubular dysfunction leading to decreased creatinine clearance, and increased BUN and creatinine levels in the serum. Moreover, it has been shown that the insufficient activation of mitophagy is associated with worse outcome in critically ill patients (See, Gunst J. et al. 2013). In contrast, stimulation of autophagy which is a cellular process related to mitophagy has been shown to be effective at protecting organ function.

One of the reasons for the kidney-protective role of mitophagy is that this process plays a key role in the regulation of apoptosis-related processes leading to the destruction of kidney cells. Thus, compositions capable of activating mitophagy at the earliest stages of oxidative stress and reducing the activity of apoptosis-activating molecular processes are anticipated to be particularly useful.

For example, inhibitors of the Apoptosis Protein (IAP) family members are well-known regulators of autophagosome formation (See, Pötsch I. et al. 2018). A key molecular entity involved in activating autophagy in the early stages of oxidative stress-induced tissue injuries is the protein kinase C delta (also called PRKCD or PKCδ) which promotes the dissociation of Bcl-2 from the autophagy-inhibiting Bcl-2/Beclin 1 (BECN1) complex. (See, Chen J. L. et al. 2008). Thus, it is known that septic shock and polymyxin increase reactive oxygen levels which affect interactions of the protein Keap1 with the nuclear protein NFE2L2. Wherein the Keap1 protein is a detector of reactive oxygen species (ROS) and by binding to NFE2L2 inhibits its translocation into the nucleus and the expression of proteins involved in antioxidant responses leading to the down regulation of ROS production and the expression of p62 protein (also known as SQSTM1) which regulates autophagosome formation and repair of damaged cellular and mitochondrial components. When these protective processes are overwhelmed (See, Wu, H., et al. 2016), apoptotic processes are activated causing damage to kidney and brain tissues. Processes known to be involved in the induction of acute renal injury and ischemia include necroptosis and ferroptosis which are processes that are regulated in part by autophagy, mitophagy and reactive oxygen species (ROS) levels. Ferroptosis is a specialized form of autophagy-regulated cell death that is triggered by increases in lipid peroxidation and this process is promoted by the association of Beclin 1 with the protein SLC7A11 which is a component of the Cystine/Glutamate Antiporter System xc—(See, Song X. et al. 2018). Ferrostatin, liproxstatin-1, Sanglifehrin A, baicalein, aminooxyacetic acid, deferoxamine, dopamine, vitamin C, ethylenediamine, and diacerein are known inhibitors of ferroptosis (See, Linkermann A. et al. (2014).

Involvement of Beclin 1 in aminoglycoside induction of acute renal injury is implicated in observations indicating that the lipid-lowering drug atorvastatin shows protective effects against gentamicin (GM)-induced nephrotoxicity. Thus, atorvastatin activates autophagy through the MEK/ERK signaling pathway (See, Li N. et al. 2014) and activation of protein kinase C delta (See, Sassano A et al 2012) which, in turn, affects Beclin 1 association with BCL2 and the association of Beclin 1 with SLC7A11 of the system xc-complex. Concerning molecular mechanisms of how atorvastatin attenuates GM-induced acute kidney injury, atorvastatin may induce expression levels of SLC9A3R1 (See, Lee, M. C. et al. 2019) which stimulates autophagy via Beclin 1 stabilization (See, Hong Liu et al 2015). Confirming the importance of this mechanism in reducing polymyxin B- and colistin-induced acute renal injury we discovered that atorvastatin indeed blocks the elevation of creatinine and urea levels induced by the administration of 3 mg/kg/diem of colistin over a period eight days in rats (see experimental section). This observation indicates that atorvastatin has utility in reducing polymyxin B- and colistin-induced acute nephrotoxicity even though the long term use of statins, by down-regulating the expression of the protective enzyme antioxidant enzyme glutathione peroxidase 4, exacerbates kidney injury (See, Verdoodt, A. et al. 2018).

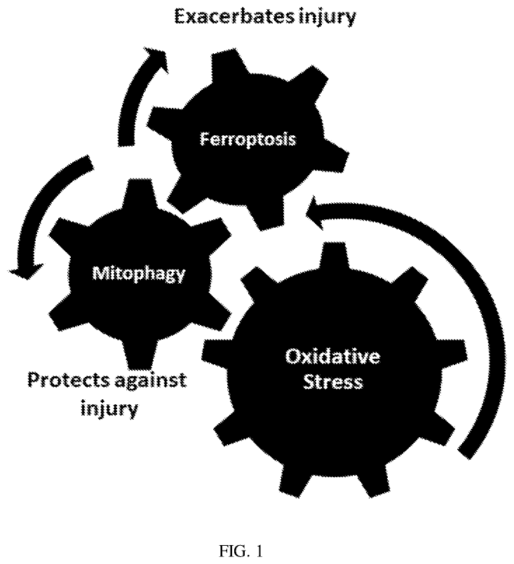

Thus, for taking advantage of the kidney-protective effects of mitophagy against polymyxin-induced acute kidney injury it is desirable to identify pharmacological agents that are capable of not only activating mitophagy at the early stages of oxidative stress but also capable of inhibiting ferroptosis and lipid peroxidation which accelerate generation of tissue injury (See FIG. 1).

Compositions, combinations and methods of the present disclosure are particularly useful for preventing polymyxin B- and colistin-induced acute renal toxicity and, in so doing, enable treatment of life-threatening bacterial infections (See, Ghlissi, Z. et al. 2018).

The nephrotoxicity of cisplatin is multifactorial and involves necroptosis (See, Kim J. et. al. 2012; Xu Y. et al. 2015). This process leads to the alteration in the number and size of lysosomes and mitochondria, disruption of the cytoskeletal integrity, cell polarity, loss of brush border, mislocalization of the sodium/potassium ATPase, decreased number of aquaporin water channels (AQP2 and AQP3 in collecting duct and AQP1 in proximal nephron and renal microvasculature), which are jointly responsible for cisplatin-induced urinary concentration defects. Depending on the dosage, cisplatin may lead to cell injury or cell death, i.e., autophagy, apoptosis, and necrosis (See, Perše, M. et al. 2018). Thus, it is known that the 5-lipoxygenase inhibitor zileuton (See, Helmy, M. M. et al. 2018), and the radical scavengers edaravone (See, Satoh M. 2003), protect rats against cisplatin-induced renal damage; however, these experiments do not refer to zileuton or edaravone protecting against polymyxin B- or colistin-induced kidney injury.

Aspects of the disclosed embodiments are directed to pharmaceutical compositions, pharmaceutical combinations and methods of treatment including zileuton, edaravone and atorvastatin compounds combined with nephrotoxicity- and neurotoxicity-inducing antibiotic or anticancer drugs for treating bacterial infections and cancers. A second aspect of the disclosed embodiments relates to the use of zileuton, edaravone and atorvastatin either alone or in combination for treatment of acute renal injury. A third aspect of the disclosed embodiments relates to the use of zileuton either alone or in combination with atorvastatin and edaravone at a daily dose ranging from about 30 mg/kg to about 180 mg/kg for treatment of neurodegenerative diseases including Alzheimer's disease and other dementias, Parkinson's disease (PD) and PD-related disorders, prion disease, motor neuron diseases (MND), Huntington's disease (HD), spinocerebellar ataxia (SCA), spinal muscular atrophy (SMA), amyotrophic lateral sclerosis (ALS).

Herein we have discovered that even very low doses of zileuton, (known to be capable of activating protein kinase C delta and autophagy and of inhibiting ferroptosis (See, Hyun-Jeong K. et al. 2010), produce potent inhibition of colistin-induced acute renal injury in rat and mouse models of renal injury. Moreover, combinations of polymyxin B and colistin with zileuton are anticipated to be particularly useful for treatment of bacterial infections in patients with and without sepsis.

Neurotoxicity remains the other unwanted side-effect of polymyxins. Thus, administration of 15 mg/kg/day of colistin for 7 days in mice induced mitochondrial dysfunction in central and peripheral nerve tissues (See, Dai, Li, & Li, 2013). In addition, mouse N2a neuronal cells treated with 200 μM colistin for 24 h become apoptotic (See, Dai, Ciccotosto, et al., 2016). Using immunohistochemistry and Western blotting, it was identified that colistin-induced apoptosis in N2a neuronal cells involves the generation of reactive oxygen species (ROS) (See, Dai, et al., 2017). Inhibition of autophagy by chloroquine enhances colistin-induced apoptosis (See, Dai, Ciccotosto, et al., 2016). Moreover, colistin treatment induces the activation of pro-inflammatory mediators (NF-kappa B, COX-2 and IL-1β) in neuronal cells (See, Dai, Ciccotosto, et al., 2016).

Thus, compositions of the present disclosure may modulate interactions between proteins identified in Table 1 and be useful for mitigating sepsis but also for reducing renal and neuronal injury induced by various toxins and disease-causing events; however, the most preferred embodiments aspects of the present disclosure also include drug combinations for treatment of bacterial infections with reduced renal and neurotoxicity including the anti-inflammatory drug zileuton and natural and synthetic polymyxin antibiotics (such as for example, MRX-8, SPR741, SPR206, CA824, FADDI-002, FADDI-003, FADDI-287, MICuRx-12, NAB739, NAB815 and octapeptin C4), bacitracin, aminoglycosides (such as for example, apramycin, plazomicin, neomycin, kanamycin, paromomycin, spectinomycin, gentamicin), amphotericin B and tetracyclines either alone or in combination with amikacin, azithromycin, aztreonam, beta-lactam/beta lactamase inhibitors (such as for example, ceftazidime-avibactam, ceftolozane-tazobactam, imipenem-relebactam, meropenem-vaborbactam), chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracyclines, tigecycline, doxycycline, minocycline, trimethoprim-sulfamethoxazole, vancomycin.

The chemical zileuton, contains one weakly acidic hydrogen atom and one asymmetric center and thus gives rise to the formation of salt forms and enantiomers that may be defined, in terms of absolute stereochemistry, as (R)- or (S)- and in terms of their optical rotations as (+) and (−) enantiomers. The present disclosure is meant to include all salt forms and racemic mixtures, optically pure forms and cyclodextrin derived inclusion complex mixtures. The sodium salt of zileuton is commercially available and the optically active (R)- and (S)-isomers may be prepared using known chiral synthons, chiral reagents, or separated into pure enantiomers using other means known in the art. “Stereoisomers” in turn, are isomers that differ only in the way the atoms are arranged in space. “Enantiomers” are a pair of stereoisomers that are non-superimposable mirror images of each other. A 1:1 mixture of a pair of enantiomers is called a “racemic” mixture. The term “(±)” is used to designate a racemic mixture where appropriate. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. It is intended that the compounds described herein include racemates, both E and Z geometric isomers and various pharmaceutically acceptable salt forms. As used, herein, the term “zileuton” encompasses ((±) 1-[1-(1-benzothiophen-2-yl)ethyl]-1-hydroxyurea, the optically pure form of the (S)-enantiomer or (−)-isomer of N-(l-benzo[b]thien-2-ylethyl)-N-hydroxyurea (as described, for example, in U.S. Pat. No. 5,629,337, which is incorporated by reference herein in its entirety), the optically pure form of (R)-enantiomer or (+)-isomer of N-(l-benzo[b]thien-2-ylethyl)-N-hydroxyurea (as described, for example, in WO 94/26268) and mixtures of said (S)- and (R)-isomers in any ratio between 1:99 and 99:1, and polymorphic forms of zileuton that are now known or later discovered Preferred salts are the sodium salt of zileuton (sodium; 1-[1-(1-benzothiophen-2-yl)ethyl]-1-oxidourea)

As used, herein, the term “edaravone” encompasses 5-methyl-2-phenyl-4H-pyrazol-3-one and all pharmaceutically acceptable salts as well as all racemic mixtures, optically pure forms thereof.

As used, herein, the term “atorvastatin” encompasses 3R,5R)-7-[2-(4-fluorophenyl)-3-phenyl-4-(phenylcarbamoyl)-5-propan-2-ylpyrrol-1-yl]-3,5-dihydroxyheptanoic acid and all pharmaceutically acceptable salts as well as all racemic mixtures, optically pure forms thereof. Preferred salts are the calcium salt calcium; (3R,5R)-7-[2-(4-fluorophenyl)-3-phenyl-4-(phenylcarbamoyl)-5-propan-2-ylpyrrol-1-yl]-3,5-dihydroxyheptanoate and the sodium salt sodium; (3S,5S)-7-[2-(4-fluorophenyl)-3-phenyl-4-(phenylcarbamoyl)-5-propan-2-ylpyrrol-1-yl]-3,5-dihydroxyheptanoate.

Formulations, methods of use or treatment and pharmaceutical compositions of the present disclosure include enabling pulmonary, intramuscular (IM), subcutaneous (SC), intravenous (IV), intrathecal and intraventricular delivery of kidney-protective amounts of zileuton, edaravone and atorvastatin, preferably zileuton, in combination with IV-injected antibacterials, for example, for example, polymyxins B and E, plazomicin, neomycin, kanamycin, paromomycin, spectinomycin, bacitracin, gentamicin, amphotericin B and tetracyclines are expected to have a significant advantage for treatment of bacterial infections and permit treatment of a broad range of infectious disease states. In addition, it is desirable to deliver these formulations and pharmaceutical compositions in a physiologically acceptable carrier (also referred to as a pharmaceutically acceptable carrier). For example, it is known that one may inject a compound into a patient in a pharmaceutically acceptable carrier, such as for example, buffered saline solution. Methods as well as combinations and pharmaceutical compositions of the present disclosure and individual components thereof can be administered by injection into a patient in a pharmaceutically acceptable carrier, such as for example, buffered saline solution. Injection into an individual may occur intramuscular, subcutaneous, intravenously, intrathecal and intraventricular or, if pulmonary delivery is desired, by use of an aerosol.

Towards this end WO0232459 (A2) with priority date Oct. 17, 2000 describes methods of increasing the biological activity of a bioactive agent by complexing the bioactive agent with a complexing agent. In one preferred embodiment, the bioactive agent is an antibiotic including colistin but not polymyxin B and the complexing agent is a cyclodextrin including hydroxypropyl-p-cyclodextrin. Furthermore, WO0232459 relates that this method may be extended to include any drugs as bioactive agents and that those skilled in the art will appreciate that both natural and chemically modified cyclodextrins are readily available in the art and may be used in embodiments of the present disclosure to increase the biological activity of a bioactive agent (See “Comprehensive Supramolecular Chemistry” Volume 3, edited by József Szejtili and Tetsuo Osa, published by Elsevier Science Inc., New York, N.Y.). Naturally occurring cyclodextrins include a-, (3-, and y-cyclodextrins (See, Pagington, 1987); Parrish, Cyclodextrins-A Review, Sterling Organics Ltd. Newcastle-Upon-Tyne. England; Szejtli, Cyclodextrin Technology. Topics in Inclusion Science, Kluwer Academic Publishers 1988). Furthermore, WO0232459 describes that the hydroxypropyl-p-cyclodextrin system is a highly complex mixture of various isomeric forms of variously substituted β-cyclodextrin derivatives conveying amorphousness which has beneficial effects on aqueous solubility and toxicity (See, Müller et al., (1985) Pharm Res. 10: 309).

Furthermore, WO2007059507 refers to water soluble formulations comprising an inclusion complex of therapeutically effective concentrations of a lipoxygenase inhibitor such as for example, zileuton with a B-cyclodextrin and pharmaceutically acceptable excipients. In addition, WO2007059507 refers to a method of making an aqueous solution of an inclusion complex of a 5-lipoxygenase inhibitor and a [beta]-cyclodextrin such as 2-hydroxypropyl-[beta]-cyclodextrin comprising the steps of: preparing an aqueous buffer solution; dissolving the [beta]-cyclodextrin derivative in the buffer solution; and adding a 5-lipoxygenase inhibitor to the [beta]-cyclodextrin derivative and buffer solution. Furthermore, WO2007059507 refers to a method of treating a mammal suffering from a condition mediated by lipoxygenase and/or leukotriene activity by administering the pharmaceutical composition comprising a lipoxygenase inhibitor and a cyclodextrin wherein said lipoxygenase inhibitor is present at a therapeutically effective concentration of the lipoxygenase inhibitor.

Concerning the range of therapeutic effective concentrations U.S. Pat. Nos. 4,873,259, 4,992,464, and 5,250,565 which are incorporated herein by reference in their entirety, refer to 5- and/or 12-lipoxygenase inhibiting compounds including zileuton, pharmaceutical formulations of said inhibitors and that a solid dosage form of 600 mg zileuton is used as a treatment for asthma. Zileuton may be used as a racemic mixture (about 50:50) of R(+) and S(−) enantiomers. Isomers of zileuton have also been described. U.S. Pat. No. 5,629,337, which is incorporated herein by reference in its entirety refers to the use of optically pure (−)-zileuton and WO 94/26268, which is incorporated herein by reference discloses the use of optically pure (+)-zileuton. U.S. Pat. No. 5,629,337 (A) relates that in pre-clinical trials, racemic zileuton was absorbed rapidly in all the species tested with Tₘₐₓ values ranging from 15 minutes to one hour and that the elimination half-life estimated from oral studies, varied markedly among species from 20 minutes in monkeys to 7 hours in dogs. Furthermore U.S. Pat. No. 5,629,337 (A) refers to therapeutic dose ranges for (−)-zileuton in the acute or chronic management of disease and US2010273868 (A1) refers to therapeutic dose ranges for the (−) enantiomer. Both of these patents relate that therapeutic efficacious doses of zileuton vary according to the age, body weight and response of the individual patient and disclose in general terms that therapeutically effective daily dose of (+), (−) and racemic zileuton range from about 200 mg to about 2 g in single or divided doses wherein the preferable daily dose range is about 400 mg to about 1600 mg in single or divided doses and the most preferred daily dose range about 600 mg to about 1200 mg in single or divided doses. WO2007059507 also refers to intravenous push formulations, the concentration of zileuton would have to be high enough to provide a dosage that causes an ameliorative effect without having to administer more than the typical maximum volume for an I.V. push of about 100 mL.

Herein we unexpectedly discovered that zileuton, administered I.P. at doses of around 3 mg/kg, which is the human dose equivalent of about 0.5 mg/kg (See, J Basic Clin Pharm. March 2016-May 2016; 7(2): 27-31) blocks the elevation of creatinine and urea levels induced by a dose of 30 mg kg colistin in a rat model of acute renal injury. Thus, the therapeutically effective dose of zileuton in a rat model of acute renal injury is well below the 1480 mg/kg dose, which in the rat is the human dose equivalent of a 200 mg/kg which is the lowest reported therapeutic effective dose of zileuton.

An embodiment of the present disclosure includes a method of treating bacterial infections in a mammal comprising administering to said mammal in need of such treatment an effective amount of zileuton or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methansulfonate, sodium colistimethate, MRX-8, SPR741, SPR206, CA824, FADDI-002, FADDI-003, FADDI-287, MICuRx-12, NAB739, NAB815 and octapeptin C4. The aforementioned method embodiment can include a method of treating sepsis. The aforementioned method embodiment may optionally further include administering an effective amount of one or more antibiotics selected from the group consisting of amikacin, apramycin, azithromycin, aztreonam, menopenem-vaborbactam, imipenem-relebactam, ceftazidime-avibactam, ceftolozane-tazobactam, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin. The aforementioned method embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methansulfonate may be an intravenous pharmaceutical composition. The aforementioned method embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methanesulfonate and sodium colistimethate may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg. The aforementioned method embodiment wherein one or more nephrotoxicity- or neurotoxicity-inducing antibiotics is selected from the group consisting of colistin, colistin sulfomethate, colistin methanesulfate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg and further including a beta cyclodextrin derivative in amounts that are sufficient to solubilize said antibiotics and said zileuton. The aforementioned method embodiment may optionally further include one or more antibiotics selected from the group consisting of amikacin, apramycin, azithromycin, aztreonam, menopenem-vaborbactam, imipenem-relebactam, ceftazidime-avibactam, ceftolozane-tazobactam, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin. The aforementioned method of administering an effective amount of zileuton or a pharmaceutically acceptable salt thereof and an effective amount of a nephrotoxicity- or neurotoxicity-inducing antibiotic may optionally include oral, pulmonary and parenteral drug administration. The common parenteral routes are intramuscular (IM), subcutaneous (SC) and intravenous (IV).

An embodiment of the present disclosure includes a combination comprising (a) zileuton or a pharmaceutically acceptable salt thereof, preferably an effective amount thereof, and (b) one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815 and octapeptin C4. The aforementioned combination embodiment may optionally further include one or more antibiotics selected from the group consisting of amikacin, apramycin, azithromycin, aztreonam, menopenem-vaborbactam, imipenem-relebactam, ceftazidime-avibactam, ceftolozane-tazobactam, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin. The aforementioned combination embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methanesulfate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition. The aforementioned combination embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methanesulfate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg. The aforementioned combination embodiment wherein one or more nephrotoxicity- or neurotoxicity-inducing antibiotics is selected from the group consisting of colistin, colistin sulfomethate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg and further including a beta cyclodextrin derivative in amounts that are sufficient to solubilize said antibiotics and said zileuton. The aforementioned combination embodiment may optionally further include one or more antibiotics selected from the group consisting of amikacin, apramycin, apramycin, azithromycin, aztreonam, ceftazidime-avibactam, chloramphenicol, clindamycin, daptomycin, doxycycline, eravacycline, erythromycin, fosfomycin, fusidic acid, levofloxacin, linezolid, LpxC inhibitor CHIR-090, meropenem, minocycline, rifampin, spectinomycin, tetracycline, tigecycline, trimethoprim-sulfamethoxazole, vancomycin, and gentamicin.

An embodiment of the present disclosure includes a pharmaceutical composition for treating bacterial infections in a mammal comprising zileuton or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfate, sodium colistimethate, MRX-8, SPR741, SPR206, CA824, FADDI-002, FADDI-003, FADDI-287, MICuRx-12, NAB739, NAB815 and octapeptin C4; and a pharmaceutically acceptable carrier. The aforementioned pharmaceutical composition embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition. The aforementioned pharmaceutical composition embodiment wherein one or more nephrotoxicity- or neurotoxicity-inducing antibiotics is selected from the group consisting of polymyxin B and polymyxin B sulfate or is selected from the group consisting of colistin, colistin sulfomethate, colistin methanesulfate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg. The aforementioned pharmaceutical composition embodiment wherein one or more nephrotoxicity- or neurotoxicity-inducing antibiotics is selected from the group consisting of colistin, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4 may be an intravenous pharmaceutical composition, the effective amount of said one or more nephrotoxicity- or neurotoxicity-inducing antibiotics is between about 1.0 to about 25 mg CBA/kg per dose and the effective amount of zileuton is between about 0.5 mg/kg to about 180 mg/kg and further including a beta cyclodextrin derivative in amounts that are sufficient to solubilize said antibiotics and said zileuton. The aforementioned pharmaceutical composition embodiment may optionally further include one or more antibiotics selected from the group consisting of amikacin, apramycin, azithromycin, aztreonam, menopenem-vaborbactam, imipenem-relebactam, ceftazidime-avibactam, ceftolozane-tazobactam, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin.

An embodiment of the present disclosure includes a method of treating cancer in a mammal or prolonging the survival of the mammal comprising administering to said mammal in need of such treatment an effective amount of zileuton or a pharmaceutically acceptable salt thereof and an effective amount of a nephrotoxicity- or neurotoxicity-inducing anticancer drug including ifosfamide, or an immune checkpoint inhibitor such as for example, ipilimumab, pembrolizumab and nivolumab.

An embodiment of the present disclosure includes a combination comprising (a) zileuton or a pharmaceutically acceptable salt thereof, preferably an effective amount thereof, and (b) one or more nephrotoxicity- or neurotoxicity-inducing anticancer drugs, preferably an effective amount thereof, including ifosfamide, or an immune checkpoint inhibitors such as for example, Ipilimumab, pembrolizumab and nivolumab.

An embodiment of the present disclosure includes a pharmaceutical composition for treating cancer in a mammal or prolonging the survival of the mammal comprising zileuton or a pharmaceutically acceptable salt thereof, preferably an effective amount thereof; one or more nephrotoxicity- or neurotoxicity-inducing anticancer drugs, preferably an effective amount thereof, including ifosfamide or an immune checkpoint inhibitor such as for example, Ipilimumab, pembrolizumab and nivolumab; and a pharmaceutically acceptable carrier.

An embodiment of the present disclosure includes a method of treating acute kidney injury in a mammal comprising administering to said mammal in need of such treatment of an effective amount of zileuton or a pharmaceutically acceptable salt thereof.

An embodiment of the present disclosure includes a method of treating diabetic nephropathy in a mammal comprising administering to said mammal in need of such treatment of an effective amount of zileuton or a pharmaceutically acceptable salt thereof.

An embodiment of the present disclosure includes a method of treating bacterial infections in a mammal comprising administering to said mammal in need of such treatment of an effective amount of edaravone or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4. The aforementioned method embodiment can include a method of treating sepsis.

An embodiment of the present disclosure includes a combination comprising (a) edaravone or a pharmaceutically acceptable salt thereof, preferably an effective amount thereof, and (b) one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

An embodiment of the present disclosure includes a pharmaceutical composition for treating bacterial infections in a mammal comprising edaravone or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, gentamicin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4; and a pharmaceutically acceptable carrier.

An embodiment of the present disclosure includes a method of treating cancer in a mammal or prolonging the survival of the mammal comprising administering to said mammal in need of such treatment an effective amount of edaravone or a pharmaceutically acceptable salt thereof and an effective amount of a nephrotoxicity- or neurotoxicity-inducing anticancer drug including ifosfamide or an immune checkpoint inhibitor such as for example, Ipilimumab, pembrolizumab and nivolumab.

An embodiment of the present disclosure includes a combination comprising (a) edaravone or a pharmaceutically acceptable salt thereof, preferably an effective amount thereof, and (b) one or more nephrotoxicity- or neurotoxicity-inducing anticancer drugs, preferably an effective amount thereof, including ifosfamide or an immune checkpoint inhibitor such as for example, Ipilimumab, pembrolizumab and nivolumab.

An embodiment of the present disclosure includes a pharmaceutical composition for treating cancer in a mammal or prolonging the survival of the mammal comprising edaravone or a pharmaceutically acceptable salt thereof, preferably an effective amount thereof; one or more nephrotoxicity- or neurotoxicity-inducing anticancer drugs, preferably an effective amount thereof, including, ifosfamide or an immune checkpoint inhibitor such as for example, Ipilimumab, pembrolizumab and nivolumab; and a pharmaceutically acceptable carrier.

An embodiment of the present disclosure includes a method of treating bacterial infections in a mammal comprising administering to said mammal in need of such treatment of an effective amount of atorvastatin or a pharmaceutically acceptable salt thereof and an effective amount of a nephrotoxicity- or neurotoxicity-inducing antibiotic selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4. The aforementioned method embodiment can include a method of treating sepsis.

An embodiment of the present disclosure includes a combination comprising (a) atorvastatin or a pharmaceutically acceptable salt thereof, preferably an effective amount thereof, and (b) one or more nephrotoxicity- or neurotoxicity-inducing antibiotics, preferably an effective amount thereof, selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

An embodiment of the present disclosure includes a pharmaceutical composition for treating bacterial infections in a mammal comprising atorvastatin or a pharmaceutically acceptable salt thereof, preferably an effective amount thereof; one or more nephrotoxicity- or neurotoxicity-inducing antibiotics, preferably an effective amount thereof, selected from the group consisting of plazomicin, neomycin, kanamycin, paromomycin, bacitracin, polymyxin B, colistin, amphotericin B, tetracyclines, polymyxin B, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4; and a pharmaceutically acceptable carrier.

An embodiment of the present disclosure includes a method of treating cancer in a mammal or prolonging the survival of the mammal comprising administering to said mammal in need of such treatment an effective amount of atorvastatin or a pharmaceutically acceptable salt thereof and an effective amount of a nephrotoxicity- or neurotoxicity-inducing anticancer drug including ifosfamide or an immune checkpoint inhibitor such as for example, Ipilimumab, pembrolizumab and nivolumab.

An embodiment of the present disclosure includes a combination comprising (a) atorvastatin or a pharmaceutically acceptable salt thereof, preferably an effective amount thereof, and (b) one or more nephrotoxicity- or neurotoxicity-inducing anticancer drugs, preferably an effective amount thereof, including ifosfamide or an immune checkpoint inhibitor such as for example, Ipilimumab, pembrolizumab and nivolumab.

An embodiment of the present disclosure includes a pharmaceutical composition for treating cancer in a mammal or prolonging the survival of the mammal comprising atorvastatin or a pharmaceutically acceptable salt thereof, preferably an effective amount thereof; one or more nephrotoxicity- or neurotoxicity-inducing anticancer drugs, preferably an effective amount thereof, including ifosfamide or an immune checkpoint inhibitor such as for example, Ipilimumab, pembrolizumab and nivolumab; and a pharmaceutically acceptable carrier.

An effective amount of zileuton, edaravone or atorvastatin may include an amount effective to treat acute kidney injury or to protect the kidneys.

An effective amount of one or more a nephrotoxicity- or neurotoxicity-inducing antibiotics of the present disclosure may include an amount effective to treat a bacterial infection.

An effective amount of one or more nephrotoxicity- or neurotoxicity-inducing anticancer drugs of the present disclosure may include an amount effective to treat cancer in a mammal or prolonging the survival of the mammal.

Experimental

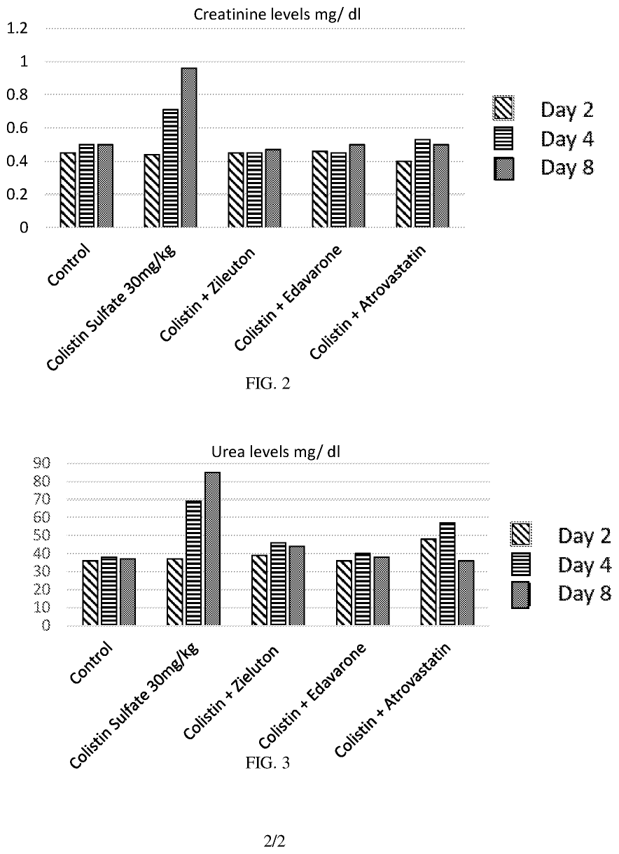

Rats were injected I.P. on the left side of the animals with a 30 mg/kg dose of colistin once a day for eight consecutive days. This injection protocol was repeated but in addition to the colistin administration each animal was also treated with an I.P. injection of (a) zileuton 3 mg/kg, (b) edaravone 15 mg/kg on the right side of the animal for 7 days and (c) P.O. administration of atorvastatin 25 mg/kg for 7 days. Effects of colistin on kidney functions were measured prior to dosing on days 1, 4 and 8 by determining the reduction of colistin-induced elevation of serum urea and creatinine levels in presence of zileuton, edaravone and atorvastatin shown in Table 2 below and in FIGS. 2 and 3.

TABLE 2

| | Mean Creatinine | | | | Mean Urea | | |
|---|---|---|---|---|---|---|---|
| | (mg/dl) | | | | (mg/dl) | | |
| | Day | Day | Day | | Day | Day | Day |
| Treatment Group | 1 | 4 | 8 | Treatment Group | 1 | 4 | 8 |
| Control | 0.45 | 0.5 | 0.5 | Control | 36 | 38 | 37 |
| Colistin Sulfate | 0.44 | 0.71 | 0.96 | Colistin Sulfate | 37 | 69 | 85 |
| Colistin + Zileuton | 0.45 | 0.45 | 0.47 | Colistin + Zileuton | 39 | 46 | 44 |
| Colistin + | 0.46 | 0.45 | 0.5 | Colistin + | 36 | 40 | 38 |
| Edavarone | | | | Edavarone | | | |
| Colistin + | 0.4 | 0.53 | 0.5 | Colistin + | 48 | 57 | 36 |
| Atrovastatin | | | | Atrovastatin | | | |

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1.-93. (canceled)

94. A method of treating bacterial infections in a mammal comprising administering to said mammal in need of such treatment an effective amount of zileuton or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

95. A combination comprising (a) zileuton or a pharmaceutically acceptable salt thereof and (b) one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, sodium colistimethate, MRX-8, SPR741, SPR206, CA824, FADDI-002, FADDI-003, FADDI-287, MICuRx-12, NAB739, NAB815 and octapeptin C4.

96. The combination according to claim 95, wherein one or more nephrotoxicity- or neurotoxicity-inducing antibiotics is selected from the group consisting of polymyxin B, polymyxin B sulfate, colistin Sulfomethate and sodium colistimethate.

97. The combination according to claim 95, wherein the one or more nephrotoxicity- or neurotoxicity-inducing antibiotics is amphotericin B.

98. The combination according to claim 95, wherein the one or more nephrotoxicity- or neurotoxicity-inducing antibiotics is paromomycin.

99. The combination according to claim 95, wherein the one or more nephrotoxicity- or neurotoxicity-inducing antibiotics is selected from the group consisting of kanamycin and gentamicin.

100. The combination according to claim 95, wherein the one or more nephrotoxicity- or neurotoxicity-inducing antibiotics is neomycin.

101. A pharmaceutical composition for treating bacterial infections in a mammal comprising an effective amount of zileuton or a pharmaceutically acceptable salt thereof; an effective amount of one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4; and a pharmaceutically acceptable carrier.

102. The pharmaceutical composition according to claim 101, wherein the pharmaceutical composition is an intravenous combination.

103. The pharmaceutical composition according to claim 101, wherein one or more nephrotoxicity- or neurotoxicity-inducing antibiotics is selected from the group consisting of polymyxin B and polymyxin B sulfate.

104. The pharmaceutical composition according to claim 101, wherein the one or more nephrotoxicity- or neurotoxicity-inducing antibiotics is paromomycin.

105. The pharmaceutical composition according to claim 101, wherein the one or more nephrotoxicity- or neurotoxicity-inducing antibiotics is selected from the group consisting of kanamycin and gentamicin.

106. The pharmaceutical composition according to claim 101, wherein the one or more nephrotoxicity- or neurotoxicity-inducing antibiotics is neomycin.

107. A method of treating cancer in a mammal or prolonging the survival of the mammal comprising administering to said mammal in need of such treatment an effective amount of zileuton or a pharmaceutically acceptable salt thereof and an effective amount of a nephrotoxicity- or neurotoxicity-inducing anticancer drug selected from the group consisting of ifosfamide, ipilimumab, pembrolizumab and nivolumab.

108. A combination comprising (a) zileuton or a pharmaceutically acceptable salt thereof and (b) one or more nephrotoxicity- or neurotoxicity-inducing anticancer drugs selected from the group consisting of ifosfamide, ipilimumab, pembrolizumab and nivolumab.

109. A pharmaceutical composition for treating cancer in a mammal or prolonging the survival of the mammal comprising an effective amount of zileuton or a pharmaceutically acceptable salt thereof; an effective amount of one or more nephrotoxicity- or neurotoxicity-inducing anticancer drugs selected from the group consisting of ifosfamide, ipilimumab, pembrolizumab and nivolumab; and a pharmaceutically acceptable carrier.

110. A method of treating acute kidney injury in a mammal comprising administering to said mammal in need of such treatment of an effective amount of zileuton or a pharmaceutically acceptable salt thereof.

111. A method of treating diabetic nephropathy in a mammal comprising administering to said mammal in need of such treatment of an effective amount of zileuton or a pharmaceutically acceptable salt thereof.

112. A method of treating bacterial infections in a mammal comprising administering to said mammal in need of such treatment of an effective amount of edaravone or a pharmaceutically acceptable salt thereof and an effective amount of a nephrotoxicity- or neurotoxicity-inducing antibiotic selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

113. A combination comprising (a) edaravone or a pharmaceutically acceptable salt thereof and (b) one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4.

114. The combination according to claim 113, wherein one or more nephrotoxicity- or neurotoxicity-inducing antibiotics is selected from the group consisting of polymyxin B, polymyxin B sulfate, colistin, colistin sulfomethate, colistin methanesulfonate, and sodium colistimethate.

115. The combination according to claim 113, wherein the one or more nephrotoxicity- or neurotoxicity-inducing antibiotics is amphotericin B.

116. The combination according to claim 113, wherein the one or more nephrotoxicity- or neurotoxicity-inducing antibiotics is paromomycin.

117. The combination according to claim 113, wherein the one or more nephrotoxicity- or neurotoxicity-inducing antibiotics is selected from the group consisting of kanamycin and gentamicin.

118. The combination according to claim 113, wherein the one or more nephrotoxicity- or neurotoxicity-inducing antibiotics is neomycin.

119. A pharmaceutical composition for treating bacterial infections in a mammal comprising an effective amount of edaravone or a pharmaceutically acceptable salt thereof; an effective amount of one or more nephrotoxicity- or neurotoxicity-inducing antibiotics selected from the group consisting of plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815 and octapeptin C4; and a pharmaceutically acceptable carrier.

120. A method of treating cancer in a mammal or prolonging the survival of the mammal comprising administering to said mammal in need of such treatment an effective amount of edaravone or a pharmaceutically acceptable salt thereof and an effective amount of a nephrotoxicity- or neurotoxicity-inducing anticancer drug selected from the group consisting of ifosfamide, ipilimumab, pembrolizumab and nivolumab.

121. A combination comprising (a) edaravone or a pharmaceutically acceptable salt thereof and (b) one or more nephrotoxicity- or neurotoxicity-inducing anticancer drugs selected from the group consisting of ifosfamide, ipilimumab, pembrolizumab and nivolumab.

122. A pharmaceutical composition treating cancer in a mammal or prolonging the survival of the mammal comprising an effective amount of edaravone or a pharmaceutically acceptable salt thereof; an effective amount of one or more nephrotoxicity- or neurotoxicity-inducing anticancer drugs selected from the group consisting of ifosfamide, ipilimumab, pembrolizumab and nivolumab; and a pharmaceutically acceptable carrier.
